SEQUENCE LISTING (11 sequences)

SEQ ID NO: 1 - DNA, 1414 bp, Homo sapiens
  advanced glycosylation end product-specific receptor (AGER), transcript variant 1; NM_001136.3; GI26787960
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttga

SEQ ID NO: 2 - DNA, 1259 bp, Homo sapiens
  advanced glycosylation end product-specific receptor (AGER), transcript variant 2; NM_172197.1; GI26787961
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatgtgagtgacctggagagaggggctgggagaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttga

SEQ ID NO: 3 - DNA, 1204 bp, Homo sapiens
  advanced glycosylation end product-specific deletion exon 3 variant (AGER); AY755624.1; GI59799503
aggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatccca

SEQ ID NO: 4 - protein, 404 aa, Homo sapiens
  advanced glycosylation end product-specific receptor (AGER), transcript variant 1; NP_001127.1; GI10835203
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 5 - protein, 342 aa, Homo sapiens
  advanced glycosylation end product-specific receptor (AGER), transcript variant 2; NP_751947.1; GI26787962
MAAGTAVGAWVLVLSLWGAVVGAQNITARIGEPLVLKCKGAPKKPPQRLEWKLGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDVSDLERGAGRTRRGGANCRLCGRIRAGNSSPGPGDPGRPGDSRPAHWGHLVAKAATPRRGEEGPRKPGGRGGACRTESVGGT

SEQ ID NO: 6 - protein, 390 aa, Homo sapiens
  advanced glycosylation end product-specific deletion exon 3 variant (AGER); AAX07277.1; GI59799504
MAAGTAVGAWVLVLSLWGAVVGAQNITARIGEPLVLKCKGAPKKPPQRLEWKLGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 7 - DNA, 1436 bp, Homo sapiens
  advanced glycosylation end product-specific receptor cDNAclone MGC22357 IMAGE4718076; BC020669.1; GI18088362
gtccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagcccccacaatgatgattaaacacctgacacatctcaaaaaaaaaaaaaaaaaaaaaaaaaaaaaa

SEQ ID NO: 8 - protein, 404 aa, Homo sapiens; identical to SEQ ID NO: 4
  advanced glycosylation end product-specific receptor [Homo sapiens]; AAH20669.1; GI18088363

SEQ ID NO: 9 - protein, 318 aa, Homo sapiens
  extracellular region of advanced glycosylation end product-specific receptor [Homo sapiens]; AAH20669.1; GI18088363
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGT

SEQ ID NO: 10 - DNA, 35 bp, Artificial Sequence
  primer “N-terminus”
ctgacctatgcggccgctgctcaaaacatcacagc

SEQ ID NO: 11 - DNA, 50 bp, Artificial Sequence
  primer “C-terminus”
gactgaattcatcagtgatgatggtgatggtgagttcccagccctgatcc

(19) United States
(12) Patent Application Publication
(10) Pub. No.: US 2011/0082421 A1
(43) Pub. Date: Apr. 7, 2011

(54) Receptor-Based Blood Detoxification System

(76) Inventors: Guillermo A. Ameer, Chicago, IL (US); Cynthia M. Daniels, Chicago, IL (US); Stuart M. Sprague, Chicago, IL (US)

(21) Appl. No.: 12/278,189

(22) PCT Filed: Feb. 7, 2007
(86) PCT No.: PCT/US07/03392

§ 371 Date: Nov. 2, 2010

Related U.S. Application Data
(60) Provisional application No. 60/771,352, filed on Feb. 7, 2006.

(51) Int. Cl. A61M 1/36; A61M 37/00; B01J 20/24

(52) U.S. Cl. 604/93.01; 210/502.1; 604/500; 502/403

(57) ABSTRACT

The invention discloses compositions of matter and methods of using such compositions to detoxify blood and blood products. The invention as particular use in the treatment of diabetes, Alzheimer's disease, hemodialysis associated amyloidosis, and cardiovascular complications.

The United States government has certain rights to this invention pursuant to Grant Nos.R01DK063123-01A2 (NIH) and 1 F31 DK069013-01 from the National Institute of Digestive Diseases and Kidney Disorders (NIH) to Northwestern University.

### FIELD OF THE INVENTION

The invention relates to the field of systems and methods used for detoxifying blood and blood products. In particular, the invention provides a system comprising a receptor that binds to advanced glycation endproducts.

### BACKGROUND OF THE INVENTION

Diabetes Mellitus is quickly becoming a global epidemic. In the U.S. alone it has been estimated that more than seventeen million people are living with diabetes and by the year 2030 that number will increase to 30.3 million (Wild, S., et al., (2004) Diabetes Care, 27(5): 1047-1053; Zimmet, P., et al., (2001) Nature, 414(6865): 782-787. Although the links between chronic hyperglycemia and the development of diabetes-related complications have long been inferred, the biochemical pathways between hyperglycemia and the damage and functional alteration of tissue that accompany diabetes remain elusive (Skyler, J. S., (1996) Endocrinol. Metab. Clin. North Am., 25(2): 243-254).

There are four widely held beliefs about how hyperglycemia causes diabetic complications and one of them is the irreversible formation and deposition of reactive advanced glycation endproducts (AGEs). AGEs are a complex and heterogeneous group of compounds that are formed when reducing sugars such as glucose interact non-enzymatically with the free amino groups of proteins, lipids, and nucleic acids. The post-translational modifications of proteins through glycation leads to the accumulation of these proteins in the body with normal aging, and play an important role in the pathogenesis of angiopathy in diabetes, neurodegenerative diseases such as Alzheimer's disease, hemodialysis-associated amyloidosis, and cardiovascular complications. (See Nishikawa, T., et al., (2000) Kidney Int. Suppl., 77: S26-30; Raj, D. S., et al., (2000) Am. J. Kidney Dis., 35(3): 365-380; Baynes, J. W., (2001) Exp. Gerontol., 36(9): 1527-1537; Forbes, J. M., et al., (2003) J. Am. Soc. Nephrol., 14(8 Suppl. 3): S254-258; Wada, R. and S. Yagihashi, (2005) Ann. N.Y. Acad. Sci., 1043: 598-604; Misur, I., et al., (2004) Acta Diabetol., 41(4): 158-166; Pertynska-Marczewska, M., et al., (2004) Cytokine, 28(1): 35-47; Sasaki, N., et al., (1998) Am. J. Pathol., 153(4): 1149-1155; Miyata, T., et al., (1993) J. Clin. Invest., 92(3): 1243-1252; and Candido, R., et al., (2003) Circ. Res., 92(7): 785-792.)

The involvement of AGEs in the perturbation of many cellular functions led to the discovery of the receptor that was mediating these functions. The receptor for AGE (RAGE) is a member of the immunoglobulin superfamily of cell surface molecules, which comprises a diverse group of cell surface receptors and adhesion molecules. In vitro studies have shown that AGE-RAGE binding triggers the p21^{ras}/MAP kinase signaling cascade and leads to an increase in the expression of NF-κB controlled genes, including pro-inflammatory cytokines (for example, IL-1, IL-6, and TNF-α), vasoconstrictors such as endothelin-1, and adhesion molecules such as VCAM 1, which suggests that the AGE-RAGE interaction may be important at sites of inflammation through other pathways that are independent of hyperglycemia. (See Raj, D. S., et al., (2000) Am. J. Kidney Dis., 35(3): 365-380; Neeper, M., et al., (1992) J. Biol. Chem., 267(21): 14998-15004; Hunkapiller, T. and L. Hood, (1989) Adv. Immunol., 44: 1-63; and Singh, R., et al., (2001) Diabetologia, 44(2): 129-146.)

Recently, it has been shown that AGEs may be linked to the renin-angiotensin pathway (RAS), which is responsible for maintaining the homeostasis of peripheral vascular resistance and the volume and composition of body fluids. (See Coughlan, M. T., et al., (2005) Ann. N.Y. Acad. Sci., 1043: 750-758; Forbes, J. M., et al., (2002) Diabetes, 51(11): 3274-3282; Bohlender, J., et al., (2005) Ann. N.Y. Acad. Sci., 1043: 681-684; and Peach, M. J., (1977) Physiol. Rev., 57(2): 313-370.)

Other research groups have reported that excessive depositions of AGEs contribute to tissue damage via direct chemical cross-linking of structural proteins such as collagen, laminin, and fibronectin and through cell surface receptor-mediated pathways (Forbes, J. M., et al., (2003) J. Am. Soc. Nephrol., 14(8 Suppl 3): S254-258; Sasaki, N., et al., (1998) Am. J. Pathol., 153(4):1149-1155; Hammes, H. P., et al., (1999) Diabetologia, 42(5): 603-607; Wautier, J. L., (2001) Rev. Prat., 51(13): 1397-1399).

Cross-linking of structural proteins is thought to increase the stiffness of the protein matrix and protects it from proteolytic damage, thus contributing to the thickening and stiffening of the vascular basement membrane. The stiffening of arterial and arteriolar walls may also contribute to systemic hypertension and increased sheer stress that predispose individuals to endothelial injury and atherogenesis. The structural changes are accompanied by functional changes such as changes in the permeability and filtration properties of the basement membrane. Therefore, methods aimed at preventing and inhibiting the abnormal formation of AGEs or reducing their concentrations in the body could have a high impact upon the management of side effects due to complications from diseases associated with elevated levels of AGEs. (See McCance, D. R., et al., (1993) J. Clin. Invest., 91(6): 2470-2478; and Paul, R. G. and A. J. Bailey, (1999) Int. J. Biochem. Cell Biol., 31(6): 653-660.)

Current treatments to reduce toxic levels of AGEs primarily focus on three approaches. The first approach uses AGE inhibitors and the soluble receptor to inhibit the formation of AGEs (see, for example, Cameron, N. E., et al., (2005) Ann. N.Y. Acad. Sci., 1043: 784-792; Forbes, J. M., et al., (2004) Diabetes, 53(7): 1813-1823; and Silacci, P., (2002) J. Hypertens., 20(8): 1483-1485).

The second approach uses AGE breakers to break AGE crosslinks, and the third uses angiotensin converting enzyme (ACE) inhibition to reduce elevated levels of AGEs (see Forbes, J. M., et al., (2002) Diabetes, 51(11): 3274-3282; Asif, M., et al., (2000) Proc. Natl. Acad. Sci. 97(6): 2809-2813; Yang, S., et al. (2003) Arch. Biochem. Biophys., 412(1): 42-46; Renke, M., et al., (2005) Scand. J. Urol. Nephrol., 39(6): 511-517; Wade, V. L. and B. L. Gleason, (2004) Ann. Pharmacother., 38(7-8): 1278-1282; and Ho Song, J., et al., (2006) Nephrol. Dial. Transplant., 21(3): 683-689). A symptomatic improvement was observed using all techniques. Unfortunately, there are concerns using these techniques to reduce elevated levels of AGEs. Studies have shown that AGE inhibitors cause the inhibition of other pathways (Cameron, N. E., et al., (2005) supra). Many of these pathways may be important for the maintenance of homeostasis. Other studies hypothesize that many patients may be unable to tolerate high enough doses of ACE inhibitors to receive maximal benefit (Renke, M., et al., (2005) supra). Finally, AGE breakers were unable to reverse the increase in crosslinking in the skin or tail collagen in diabetic rats (Yang, S., et al. (2003) supra). Therefore, a new approach is needed, one that is capable of reducing elevated levels of AGEs at the same time preventing the formation of AGE crosslinks.

There is therefore a need for a blood detoxification system that can be used both in vivo and ex-corpero that provides a safe and non-immunogenic procedure that clears a patient's circulatory system of advanced glycation endproducts.

### BRIEF DESCRIPTION OF THE INVENTION

The invention provides an advanced glycation endproducts receptor (RAGE) system for detoxifying blood. The system can be used in vivo and/or extracorporally to remove advanced glycation end products (AGEs) from an individual having a disease or disorder. The disease or disorder is selected from the group consisting of disorders involving the pathogenesis of angiopathy in diabetes, neurodegenerative disorders such as Alzheimer's Disease (AD), hemodialysis associated amyloidosis, cardiovascular disorders, and inflammatory responses such as activation of macrophages and monocytes. In a preferred embodiment the system comprises a composition comprising a polypeptide having RAGE activity and a substrate. In a more preferred embodiment the polypeptide is a mammalian polypeptide and the substrate is a polymeric compound. In a still more preferred embodiment the substrate is a porous material.

In one embodiment, the invention provides a polynucleotide encoding a mammalian polypeptide having RAGE activity. In a preferred embodiment the mammal is a primate. In a more preferred embodiment, the primate is a human. In another preferred embodiment the polynucleotide encodes a polypeptide that binds AGEs with a greater affinity than that of a naturally-occurring RAGE. In yet another preferred embodiment the polynucleotide encodes a polypeptide that binds AGEs with a lower affinity than that of a naturally-occurring RAGE.

In another embodiment, the invention provides a polynucleotide comprising a polynucleotide selected from the group consisting of SEQ ID NO: 1, SEQ ID NO: 2, SEQ ID NO: 3, and SEQ ID NO: 7, or the complement thereof. In another embodiment, the invention provides a polynucleotide having at least 90% identity to SEQ ID NO: 1, the polynucleotide encoding a mammalian polypeptide having RAGE activity, or the complement thereof. In another embodiment, the invention provides a polynucleotide having at least 90% identity to SEQ ID NO: 2, the polynucleotide encoding a mammalian polypeptide having RAGE activity, or the complement thereof. In another embodiment, the invention provides a polynucleotide having at least 90% identity to SEQ ID NO: 3, the polynucleotide encoding a mammalian polypeptide having RAGE activity, or the complement thereof. In another embodiment, the invention provides a polynucleotide having at least 90% identity to SEQ ID NO: 7, the polynucleotide encoding a mammalian polypeptide having RAGE activity, or the complement thereof.

In another embodiment the invention provides a polypeptide comprising a polypeptide selected from the group consisting of SEQ ID NO: 4, SEQ ID NO: 5, SEQ ID NO: 6, SEQ ID NO: 8, and SEQ ID NO: 9. In another embodiment, the invention provides a polypeptide having RAGE activity and having 95% identity to SEQ ID NO: 4. In another embodiment, the invention provides a polypeptide having RAGE activity and having 95% identity to SEQ ID NO: 5. In another embodiment, the invention provides a polypeptide having RAGE activity and having 95% identity to SEQ ID NO: 6. In another embodiment, the invention provides a polypeptide having RAGE activity and having 95% identity to SEQ ID NO: 8. In another embodiment, the invention provides a polypeptide having RAGE activity and having 95% identity to SEQ ID NO: 9.

In another embodiment, the invention provides a polypeptide having RAGE activity and having 90% identity to SEQ ID NO: 4. In another embodiment, the invention provides a polypeptide having RAGE activity and having 90% identity to SEQ ID NO: 5. In another embodiment, the invention provides a polypeptide having RAGE activity and having 90% identity to SEQ ID NO: 6. In another embodiment, the invention provides a polypeptide having RAGE activity and having 90% identity to SEQ ID NO: 8. In another embodiment, the invention provides a polypeptide having RAGE activity and having 90% identity to SEQ ID NO: 9.

In another embodiment, the invention provides a polypeptide having RAGE activity and having 80% identity to SEQ ID NO: 4. In another embodiment, the invention provides a polypeptide having RAGE activity and having 80% identity to SEQ ID NO: 5. In another embodiment, the invention provides a polypeptide having RAGE activity and having 80% identity to SEQ ID NO: 6. In another embodiment, the invention provides a polypeptide having RAGE activity and having 80% identity to SEQ ID NO: 8. In another embodiment, the invention provides a polypeptide having RAGE activity and having 80% identity to SEQ ID NO: 9.

In another embodiment, the invention provides a polypeptide having RAGE activity and having 75% identity to SEQ ID NO: 4. In another embodiment, the invention provides a polypeptide having RAGE activity and having 75% identity to SEQ ID NO: 5. In another embodiment, the invention provides a polypeptide having RAGE activity and having 75% identity to SEQ ID NO: 6. In another embodiment, the invention provides a polypeptide having RAGE activity and having 75% identity to SEQ ID NO: 8. In another embodiment, the invention provides a polypeptide having RAGE activity and having 75% identity to SEQ ID NO: 9.

In another embodiment the invention provides a polypeptide having 100% identity to a fragment of a polypeptide selected from the group consisting of SEQ ID NOs: 4, 5, 6, 8, and 9, and wherein the fragment has RAGE activity.

In another embodiment the invention provides a polypeptide having 98% identity to a fragment of a polypeptide selected from the group consisting of SEQ ID NOs: 4, 5, 6, 8, and 9, and wherein the fragment has RAGE activity.

In another embodiment the invention provides a polypeptide having 96% identity to a fragment of a polypeptide selected from the group consisting of SEQ ID NOs: 4, 5, 6, 8, and 9, and wherein the fragment has RAGE activity.

In another embodiment the invention provides a polypeptide having 95% identity to a fragment of a polypeptide selected from the group consisting of SEQ ID NOs: 4, 5, 6, 8, and 9, and wherein the fragment has RAGE activity.

In another embodiment the invention provides a polypeptide having 93% identity to a fragment of a polypeptide selected from the group consisting of SEQ ID NOs: 4, 5, 6, 8, and 9, and wherein the fragment has RAGE activity.

In another embodiment the invention provides a polypeptide having 90% identity to a fragment of a polypeptide selected from the group consisting of SEQ ID NOs: 4, 5, 6, 8, and 9, and wherein the fragment has RAGE activity.

In another embodiment the invention provides a polypeptide having 85% identity to a fragment of a polypeptide selected from the group consisting of SEQ ID NOs: 4, 5, 6, 8, and 9, and wherein the fragment has RAGE activity.

In another embodiment the invention provides a polypeptide having 80% identity to a fragment of a polypeptide selected from the group consisting of SEQ ID NOs: 4, 5, 6, 8, and 9, and wherein the fragment has RAGE activity.

In another embodiment the invention provides a polypeptide having 75% identity to a fragment of a polypeptide selected from the group consisting of SEQ ID NOs: 4, 5, 6, 8, and 9, and wherein the fragment has RAGE activity.

In a yet further embodiment the polypeptide comprises a chimeric polypeptide, the chimeric polypeptide having RAGE AGE-binding activity and AGE-degrading activity. In an alternative embodiment the invention provides a polynucleotide, the polynucleotide encoding a polypeptide having RAGE AGE-binding activity and AGE-degrading activity.

In one embodiment the polypeptide is isolated from a peptide library. In an alternative embodiment the polypeptide is encoded by a polynucleotide isolated from a polynucleotide library. In a preferred embodiment, the polynucleotide library is a mammalian tissue library. In an alternative embodiment, the polynucleotide library is isolated from a recombinant microbial organism library.

The invention also contemplates using a recombinant mammalian receptor in a system, the receptor binding a ligand in a sample under appropriate and defined binding conditions, thereby depleting the ligand from the sample.

In a second embodiment the system comprises a polypeptide having RAGE activity and a substrate, the substrate selected from the group consisting of a micro-array, a particle, a porous particle, a membrane, a mesh, a dialysis membrane, a multi-well plate, a polymeric compound, or the like, wherein the polypeptide is chemically bound to the substrate. In a preferred embodiment the substrate is a polymeric compound. In a more preferred embodiment, the polymeric compound is agarose. In a preferred embodiment the polypeptide having RAGE activity can bind AGEs reversibly under controlled conditions, thereby allowing the system of the invention to be regenerated and used multiple times. In an alternative embodiment, the polypeptide having RAGE activity can bind AGEs irreversibly.

In a preferred embodiment the polypeptide is bound to the substrate via a linker molecule, the linker molecule selected from the group consisting of a thiol group, a sulfide group, a phosphate group, a sulfate group, a cyano group, a piperidine group, an Fmoc group, and a Boc group.

The system may also comprise at least one reservoir, at least one inlet tube, at least one outlet tube, and/or at least one pump. In use, the system is reversibly connected or attached to a fluid line that is in fluid communication with the blood or circulatory system of an individual having a disease or disorder. The pump circulates the blood through the system under conditions that enhance the binding of AGEs to the RAGE polypeptide, thereby removing substantial amounts of AGEs from the blood. The blood is returned to the individual thereby improving the individual's prognosis. The system can be used in a manner and at time intervals similar to that used with dialysis devices well known to those in the art. Examples of such systems are disclosed in U.S. Pat. No. 4,863,611, herein incorporated by reference in its entirety.

The system may also be used in vivo, whereby the system is implanted within a lumen or chamber of an organ or tissue of an individual having a disease or disorder.

In another embodiment, the RAGE polypeptide is soluble in an aqueous environment. In another embodiment the RAGE polypeptide is soluble in a non-aqueous environment. In another embodiment, the RAGE polypeptide is soluble in a mixed aqueous and non-aqueous environment. In a preferred embodiment the soluble RAGE polypetide has a domain having binding affinity for an adsorbent. The adsorbent can be a compound having specific binding activity, such as an immunoglobulin or the like, or having non-specific binding activity, such as dextran sulphate, a protein having at least one PDZ domain, or the like.

The polynucleotide encoding RAGE can be, for example, the Homo sapiens advanced glycosylation end product-specific receptor (AGER), transcript variant 1; NM_{—}001136.3, GI:26787960 (SEQ ID NO: 1 encoding SEQ ID NO: 4) and/or Homo sapiens advanced glycosylation end product-specific receptor (AGER), transcript variant 2; NM_{—}172197.1, GI:26787961 (SEQ ID NO: 2 encoding SEQ ID NO: 5) and/or Homo sapiens receptor for advanced glycosylation end-products deletion exon 3 variant (AGER) mRNA, complete cds, alternatively spliced; AY755624.1, GI:59799503 (SEQ ID NO: 3 encoding SEQ ID NO: 6) and/or Homo sapiens advanced glycosylation end product-specific receptor cDNA clone; MGC:22357; IMAGE:4718076; BC020669.1; GI:18088362 (SEQ ID NO: 7 encoding SEQ ID NO: 8).

The invention further provides a method for depleting a soluble ligand from a sample of fluid from an individual having a disease or disorder, the method comprising the steps of: i) providing a sample of fluid from an individual having a disease or disorder; ii) incubating the sample with the system comprising a receptor as disclosed herein under appropriate binding conditions; iii) allowing the soluble ligand to bind the receptor and deplete the sample of soluble ligand; and iv) returning the ligand-depleted sample to the individual, thereby depleting the soluble ligand from the sample of fluid.

The invention further provides a method for treating an individual having a disease or disorder, the method comprising the steps of: i) providing a sample of fluid from an individual having a disease or disorder; ii) incubating the sample with the system comprising a receptor as disclosed herein under appropriate binding conditions; iii) allowing the soluble ligand to bind the receptor and thereby deplete the sample of soluble ligand; and iv) returning the ligand-depleted sample to the individual, thereby treating the individual having a disease or disorder. In an additional embodiment, the method further comprises the steps of: v) incubating the sample of fluid with isolated monocytes; vi) measuring the secretion of cytokine and chemokine from the monocytes; and vii) comparing the amount of measured cytokine and chemokine in the sample before depleting the ligand with the sample after depleting the ligand. In one embodiment the cytokine and chemokine is selected from the group consisting of interleukin 1 (IL-1), interleukin 2 (IL-2), interleukin 4 (IL-4), interleukin 5 (IL-5), interleukin 6 (IL-6), interleukin 7 (IL-7), interleukin 9 (IL-9), interleukin 10 (IL-10), interleukin 13 (IL-13), tumor necrosis factor α (TNF-α), interferon α (IFN-α), interferon α-II (IFN-α-II), interferon β (IFN-β), interferon γ (IFN-γ), interferon δ (IFN-δ), macrophage migration inhibitory factor (MIF), granulocyte-macrophage colony-stimulating factor (GM-CSF), granulocyte colony-stimulating factor (G-CSF), leukemia inhibitory factor (LIF), oncostatin (OSM), autocrine motility factor (AMF), lymphotoxin-α (LT-α), lymphotoxin-β (LT-β), T cell antigen gp39 (CD40L), CD27L, CD30L, FASL, 4-1BBL, OX40L, TNF-related apoptosis inducing ligand (TRAIL), platelet factor 4 (PF4), platelet basic protein (PBP), connective-tissue activating peptide III (CTAP III), β-thromboglobulin, melanoma growth stimulatory activity protein (MGSA), macrophage inflammatory protein 2 (MIP-2), pre-B cell growth stimulating factor (PBSF), monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1α (MIP-1-α), macrophage inflammatory protein 1 β (MIP-1-β), macrophage inflammatory protein 1 γ (MEP-1-γ), macrophage inflammatory protein 3 α (MIP-3-α), macrophage inflammatory protein 3 β (MIP-3-β), macrophage inflammatory protein 4 (MIP-4), macrophage inflammatory protein 5 (MIP-5), LD78 β, RANTES, SIS-epsilon (p500), thymus and activation-regulated chemokine (TARC), Eotaxin, I-309, human protein HCC-1/NCC-2, human protein HCC-3, C-reactive protein (CRP), human PTX3, and chemotactic cytokine CP-10.

### BRIEF DESCRIPTION OF THE DRAWINGS

FIG. 1 illustrates the effects of temperature, binding site regeneration, and storage in ethanol on the binding capacity of the RAGE-based bioadsorbent.

FIG. 2 illustrates the effects of lyophilization on the bioadsorbent.

FIG. 3 illustrates removal of AGEs from the plasma of diabetic and non-diabetic patients.

FIG. 4 illustrates removal of a range of pathological concentrations of AGE-BSA

FIG. 5 illustrates that incubation of bioabsorbant with AGE-BSA (ABSA) and plasma from diabetic patients down-regulates the production of pro-inflammatory cytokines (IL-1β, IL-6, and TNF-α) and the chemokine (IL-8).

### DESCRIPTION OF THE INVENTION AND PREFERRED EMBODIMENTS

In order for a receptor-based system to become a clinical reality, it must possess a sufficient binding capacity towards AGEs. It should be amenable to regeneration and sterilization without a significant loss of binding capacity, able to withstand the various processing conditions during a patient dialysis treatment and be biologically relevant. We have developed a novel high affinity bioadsorbent that is capable of removing sufficient amounts of soluble AGEs from blood. We hypothesize that the bioadsorbent can be placed in tandem with a dialyzer during a patients' weekly dialysis therapy (see Grovender, E. A., et al., (2002) AIChE, 48(10): 2357-2365; and Daniels, C. M., et al., (2005) Blood Purif., 23(4): 287-297).

This invention discloses a receptor-based blood detoxification system for the extracorporeal removal of advanced glycation endproducts. Advanced glycation endproducts (AGEs) are implicated in the pathogenesis of angiopathy in diabetes, neurodegenerative diseases such as Alzheimer's, hemodialysis associated amyloidosis, and cardiovascular complications. Abnormal concentrations of AGEs have an effect on the activation of macrophages and monocytes, cells that are key to the exacerbation or resolution of acute and chronic inflammation. We show that AGEs present in blood from diabetic patients with end-stage kidney disease activates monocytes by upregulating cytokine and chemokine secretion and that such effect can be reversed by treating the blood with an immobilized cell receptor for AGE (RAGE). The RAGE-based bioadsorbent can be regenerated multiple times, is stable under clinically relevant conditions, and can reduce pathological AGE concentrations to normal levels. The proposed novel use of bioadsorption in extracorporeal therapy should reduce the morbidity of diabetic patients undergoing dialysis and may be extended to the general diabetic population.

AGEs perturb the biochemistry of a diverse array of cellular processes. Previous studies have shown that the interaction of AGE with RAGE upregulates an inflammatory response through the recruitment of macrophages/monocytes (Yates, S. L., et al., (2000) J. Neurochem., 74(3): 1017-10125; Cheneval, D., et al., (1998) J. Biol. Chem., 273(28): 17846-17851; and Schwedler, S., et al., (2001) Kidney Int. Suppl., 78: S32-36). Normally, inflammation is a protective response to infection by the immune system that requires communication between different classes of immune cells to coordinate their actions. However, when chronic inflammation occurs, it leads to the destruction of tissues in autoimmune disorders, neurodegenerative, and cardiovascular diseases.

The data presented herein supports the use of extracorporeal bioadsorption as a new and potentially cost effective therapeutic approach to specifically remove circulating AGEs from the blood of patients undergoing hemodialysis. It can be sterilized with 20% ethanol, lyophilized, and stored at room temperature as a lyophilized product. Once it is used on patients it can be regenerated multiple times with glycine buffer and stored in 20% ethanol awaiting the next treatment of the patient. It is herein demonstrated that the bioadsorbent is able to remove pathological concentrations of AGE-BSA. The data demonstrate that all plasma samples treated with the bioadsorbent downregulated all pro-inflammatory cytokine tested (IL-1β, IL-6, TNF-α) and the chemokine IL-8, indicating that this approach can regulate pro-inflammatory and angiogenic processes both in vivo and in vitro, possibly preventing the progression of various pathological diseases. The clinical use of agarose-immobilized receptors for the extracorporeal removal of antigens from blood is feasible and potentially cost effective and should enjoy significant commercial success.

The term “fragment” as used herein describes a portion, a region, and/or a domain of a molecule, such as, for example, a polynucleotide molecule or a polypeptide molecule. The fragment can be a portion, a region, and/or a domain of the molecule as disclosed herein. Such domains, regions and portions of a polypeptide and/or protein are well known to those of skill in the art and can include, but are not limited to, extracellular domains, transmembrane domains, intracellular domains, enzyme active catalytic sites, protein-protein interacting domains, protein-phospholipid interacting domains, polynucleotide-binding domains, and the like. The fragment can have RAGE activity. The fragment can have AGE-binding activity. The fragment can be bound to a substrate.

The term “ligand” as used herein can be used to describe any molecule and/or compound that binds to a receptor and/or bioadsorbent of the invention. The ligand can be naturally-occurring, it can be a native ligand of the receptor, it can be a synthetic ligand of the receptor, or the like.

The invention also contemplates using a recombinant mammalian receptor in a system, the receptor binding a ligand in a sample under appropriate and defined binding conditions, thereby depleting the ligand from the sample. The recombinant mammalian receptor can be a fragment of a polypeptide, the polypeptide selected from the group consisting of SEQ ID NOs: 4, 5, 6, 8, and 9, and wherein the fragment has RAGE activity.

In addition, the invention contemplates that variants of the polypeptides and fragments thereof can be used to detoxify blood or blood products. Such variants can incorporate alternative amino acid sequences in the polypeptide that do not result in loss of RAGE activity and/or AGE-binding activity. Substitution of amino acids in a polypeptide sequence, either by replacing codons or replacing amino acid residues during peptide synthesis, are well known to those of skill in the art. Such variants are desirable since the encoded polypeptide can have a different binding affinity for AGEs that a naturally-occurring or native polypeptide. The binding affinity may be less than that of the native peptide or it may be more than that of a naturally-occurring or native peptide. In addition, the polypeptide may have additional activity, such as, but not limited to, catalytic activity upon the AGEs, AGE-degrading activity, and the like, such that the polypeptide can be used to not only bind the AGEs present in a sample but also degrade and/or remove them as biologically active compounds. Methods for determining binding affinity are well known to those of skill in the art.

RAGE activity is measured using binding assays well known to those of skill in the art. Binding affinity is expressed as an association constant, Kₐ, which is defined as the molar concentration of RAGE-AGE complex divided by the molar concentrations of free AGE and RAGE under equilibrium conditions. RAGE preparations with Kₐ ranging from about 10⁶ to 10¹² l/mole are preferred.

A “variant” of a particular polynucleotide sequence is defined as a polynucleotide sequence having at least 40% sequence identity to the particular polynucleotide sequence over a certain length of one of the polynucleotide sequences using blastn with the “BLAST 2 Sequences” tool Version 2.0.9 (May 7, 1999) set at default parameters. Such a pair of polynucleotides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

For example, Table 1 illustrates, for example, that the codons AGC, AGT, TCA, TCC, TCG, and TCT all encode the same amino acid: serine. Accordingly, at each position in the sequence where there is a codon encoding serine, any of the above trinucleotide sequences can be used without altering the encoded polypeptide.

TABLE 1

| Amino acid | | | Possible Codons |
|---|---|---|---|
| Alanine | Ala | A | GCA GCC GCG GCU |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC AGT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide, these conservative variants are, likewise, a feature of the invention.

For example, substitutions, deletions and insertions introduced into the sequences provided in the Sequence Listing are also envisioned by the invention. Such sequence modifications can be engineered into a sequence by site-directed mutagenesis (Wu (ed.) Meth. Enzymol. (1993) vol. 217, Academic Press) or the other methods noted below. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 2 when it is desired to maintain the activity of the protein. Table 2 shows amino acids which can be substituted for an amino acid in a protein and which are typically regarded as conservative substitutions.

TABLE 2

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as structural and functional substitutions. For example, a residue in column 1 of Table 3 may be substituted with residue in column 2; in addition, a residue in column 2 of Table 3 may be substituted with the residue of column 1.

TABLE 3

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cys | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 2 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine.

Further Modifying Sequences of the Invention—Mutation/Forced Evolution

In addition to generating silent or conservative substitutions as noted, above, the present invention optionally includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, for example, according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel, supra, provides additional details on mutagenesis methods. Artificial forced evolution methods are described, for example, by Stemmer (1994) Nature 370:389-391, Stemmer (1994) Proc. Natl. Acad. Sci. 91:10747-10751, and U.S. Pat. Nos. 5,811,238, 5,837,500, and 6,242,568. Methods for engineering synthetic transcription factors and other polypeptides are described, for example, by Zhang et al. (2000) J. Biol. Chem. 275:33850-33860, Liu et al. (2001) J. Biol. Chem. 276:11323-11334, and Isalan et al. (2001) Nature Biotechnol. 19:656-660. Many other mutation and evolution methods are also available and expected to be within the skill of the practitioner.

Similarly, chemical or enzymatic alteration of expressed nucleic acids and polypeptides can be performed by standard methods. For example, sequence can be modified by addition of lipids, sugars, peptides, organic or inorganic compounds, by the inclusion of modified nucleotides or amino acids, or the like. For example, protein modification techniques are illustrated in Ausubel, supra. Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, e.g., using the sequences herein as a starting substrate for the various modification approaches.

For example, optimized coding sequence containing codons preferred by a particular prokaryotic or eukaryotic host can be used e.g., to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for S. cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations which modify the sequence to facilitate cloning, processing and/or expression of the gene product. For example, alterations are optionally introduced using techniques which are well known in the art, for example, site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP16 or GAL4 (Moore et al. (1998) Proc. Natl. Acad. Sci. 95: 376-381; and Aoyama et al. (1995) Plant Cell 7: 1773-1785), peptides derived from bacterial sequences (Ma and Ptashne (1987) Cell 51: 113-119) and synthetic peptides (Giniger and Ptashne, (1987) Nature 330: 670-672).

### Expression and Modification of Polypeptides

Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homologue.

The invention also contemplates a system, the system comprising a polypeptide having RAGE activity and a substrate, the substrate selected from the group consisting of a micro-array, a particle, a porous particle, a membrane, a mesh, a dialysis membrane, a multi-well plate, a polymeric compound, or the like, wherein the polypeptide is chemically bound to the substrate. In a preferred embodiment the RAGE can bind AGEs reversibly under controlled conditions, thereby allowing the system of the invention to be regenerated and used multiple times. The system can be a device for use in a clinical setting, such as a clinic or hospital, or can be used outside a building, such as when in use in the field.

In a preferred embodiment the polypeptide is bound to the substrate via a linker molecule, the linker molecule selected from the group consisting of a thiol group, a sulfide group, a phosphate group, a sulfate group, a cyano group, a piperidine group, an Fmoc group, and a Boc group. Methods for forming such linkages are well known to those of skill in the art (see, for example, Glasser, et al., (1987) Proc. Natl. Acad. Sci. 84:4007, 1987 Jacobs, et al., J. Biol. Chem. 262:9808; Floros, et al., (1986) J. Biol. Chem. 261:9029; White, et al., (1985) Nature 317:361; Glasser, et al., (1988a) J. Biol. Chem. 263:9; Glasser, et al., (1988b) J. Biol. Chem. 263: 10326; and Jobe et al., (1987) Am. Rev. Resp. Dis. 136:1032). However, polypeptides can be synthesized on a 0.25 mmol scale with an Applied Biosystems model 431 A peptide synthesizer using a FASTMOC strategy (see Fields, C. G et al., (1991) Peptide Res. 4: 95-101). The peptides can be synthesized with prederivatized Fmoc-Gly resin (Calbiochem-Nova, La Jolla, Calif.) or PEG-PA resin (Perceptive Biosystems, Old Connecticut Path, Mass.) and can be single coupled for all residues.

The system may also comprise at least one reservoir, at least one inlet tube, at least one outlet tube, and/or at least one pump. In use, the system is reversibly connected or attached to a fluid line that is in fluid communication with the blood or circulatory system of an individual having a disease or disorder. The pump circulates the blood through the system under conditions that enhance the binding of AGEs to the RAGE polypeptide, thereby removing substantial amounts of AGEs from the blood. The blood is returned to the individual thereby improving the individual's prognosis. The system can be used in a manner and at time intervals similar to that used with dialysis devices well known to those in the art.

The system may also be used in vivo, whereby the system is implanted within a lumen or chamber of an organ or tissue of an individual having a disease or disorder.

In another embodiment, the RAGE polypeptide is soluble in an aqueous environment. In another embodiment the RAGE polypeptide is soluble in a non-aqueous environment. In another embodiment, the RAGE polypeptide is soluble in a mixed aqueous and non-aqueous environment. In a preferred embodiment the soluble RAGE polypetide has a domain having binding affinity for an adsorbent. The adsorbent can be a compound having specific binding activity, such as an immunoglobulin or the like, or having non-specific binding activity, such as dextran sulphate, a protein having at least one PDZ domain, or the like.

The polynucleotide encoding RAGE can be, for example, the Homo sapiens advanced glycosylation end product-specific receptor (AGER), transcript variant 1; NM_{—}001136.3, GI:26787960 (SEQ ID NO: 1 encoding SEQ ID NO: 4) and/or Homo sapiens advanced glycosylation end product-specific receptor (AGER), transcript variant 2; NM_{—}172197.1, GI:26787961 (SEQ ID NO: 2 encoding SEQ ID NO: 5) and/or Homo sapiens receptor for advanced glycosylation end-products deletion exon 3 variant (AGER) mRNA, complete cds, alternatively spliced; AY755624.1, GI:59799503 (SEQ ID NO: 3 encoding SEQ ID NO: 6) and/or Homo sapiens advanced glycosylation end product-specific receptor cDNA clone; MGC:22357; IMAGE:4718076; BC020669.1; GI:18088362 (SEQ ID NO: 7 encoding SEQ ID NO: 8).

RAGE activity is measured using binding assays well known to those of skill in the art. Binding affinity is expressed as an association constant, Kₐ, which is defined as the molar concentration of RAGE-AGE complex divided by the molar concentrations of free AGE and RAGE under equilibrium conditions. Various methods such as Scatchard analysis in conjunction with radioimmunoassay techniques may be used to assess the affinity of RAGE for AGEs. RAGE preparations with Kₐ ranging from about 10⁶ to 10¹² l/mole are preferred. RAGE activity can also be measured using nitrocellulose filter binding assays, such as describe by Wilton et al (Wilton (2006) supra).

AGE concentrations ([AGE]) in a sample can be measured using methods well known to those of skill in the art. In one example, [AGE] is measured by incubating a sample comprising AGEs with isolated human THP-1 monocytes and measuring the amounts of cytokines and chemokines secreted from the monocytes as described by Pertynska-Marczewska et al. (2004, Cytokine 28: 35-47).

The invention further provides a method for depleting a soluble ligand from a sample of fluid from an individual having a disease or disorder, the method comprising the steps of: i) providing a sample of fluid from an individual having a disease or disorder; ii) incubating the sample with the system comprising a receptor as disclosed herein under appropriate binding conditions; iii) allowing the soluble ligand to bind the receptor and deplete the sample of soluble ligand; and iv) returning the ligand-depleted sample to the individual, thereby depleting the soluble ligand from the sample of fluid.

The invention further provides a method for treating an individual having a disease or disorder, the method comprising the steps of: i) providing a sample of fluid from an individual having a disease or disorder; ii) incubating the sample with the system comprising a receptor as disclosed herein under appropriate binding conditions; iii) allowing the soluble ligand to bind the receptor and thereby deplete the sample of soluble ligand; and iv) returning the ligand-depleted sample to the individual, thereby treating the individual having a disease or disorder. In an additional embodiment, the method further comprises the steps of: v) incubating the sample of fluid with isolated monocytes; vi) measuring the secretion of cytokine and chemokine from the monocytes; and vii) comparing the amount of measured cytokine and chemokine in the sample before depleting the ligand with the sample after depleting the ligand. In one embodiment the cytokine and chemokine are selected from the group consisting of interleukin 1 (IL-1), interleukin 2 (IL-2), interleukin 4 (IL-4), interleukin 5 (IL-5), interleukin 6 (IL-6), interleukin 7 (IL-7), interleukin 9 (IL-9), interleukin 10 (IL-10), interleukin 13 (IL-13), tumor necrosis factor α (TNF-α), interferon α (IFN-α), interferon α-II (IFN-α-II), interferon β (IFN-β), interferon γ (IFN-γ), interferon δ (IFN-δ), macrophage migration inhibitory factor (MIF), granulocyte-macrophage colony-stimulating factor (GM-CSF), granulocyte colony-stimulating factor (G-CSF), leukemia inhibitory factor (LIF), oncostatin (OSM), autocrine motility factor (AMF), lymphotoxin-α (LT-α), lymphotoxin-β (LT-β), T cell antigen gp39 (CD40L), CD27L, CD30L, FASL, 4-1BBL, OX40L, TNF-related apoptosis inducing ligand (TRAIL), platelet factor 4 (PF4), platelet basic protein (PBP), connective-tissue activating peptide III (CTAP III), β-thromboglobulin, melanoma growth stimulatory activity protein (MGSA), macrophage inflammatory protein 2 (MIP-2), pre-B cell growth stimulating factor (PBSF), monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1 α (MIP-1-α), macrophage inflammatory protein 1 β (MIP-1-β), macrophage inflammatory protein 1 γ (MIP-1-γ), macrophage inflammatory protein 3 α (MIP-3-α), macrophage inflammatory protein 3 β(MIP-3-β), macrophage inflammatory protein 4 (MIP-4), macrophage inflammatory protein 5 (MIP-5), LD78 β, RANTES, SIS-epsilon (p500), thymus and activation-regulated chemokine (TARC), Eotaxin, I-309, human protein HCC-1/NCC-2, human protein HCC-3, C-reactive protein (CRP), human PTX3, and chemotactic cytokine CP-10.

### Treatment of Disease, Disorder, or Condition.

In a further embodiment, a bioadsorbent of the invention may be used to treat or prevent a disease, disorder, or condition in an individual. Such diseases, disorders, or conditions may include, but are not limited to, Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermayitis, Crohn's disease, atopic dermatitis, dermatomyositis, diabetes mellitus, emphysema, erythema nodosum, atrophic gastritis, glomerulonephritis, Goodpasture's syndrome, gout, Graves' disease, Hashimoto's thyroiditis, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, systemic anaphylaxis, systemic lupus erythematosus, systemic sclerosis, ulcerative colitis, Werner syndrome, and complications of cancer, hemodialysis, and extracorporeal circulation; viral, bacterial, fungal, parasitic, protozoal, and helminthic infections; and trauma; akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder; adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

### Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc(9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N,N-dimethylformamide. The peptide is cleaved between the peptide carboxy terminus and the linker group to yield a peptide acid or amide. (Novabiochem 1997/98 Catalog and Peptide Synthesis Handbook, San Diego Cailf. pp. S1-20). Automated synthesis may also be carried out on machines such as the ABI 431A peptide synthesizer (PE Biosystems). A protein or portion thereof may be substantially purified by preparative high performance liquid chromatography and its composition confirmed by amino acid analysis or by sequencing (Creighton (1984) Proteins, Structures and Molecular Properties, WH Freeman, New York N.Y.).

The invention will be more readily understood by reference to the following examples, which are included merely for purposes of illustration of certain aspects and embodiments of the present invention and not as limitations.

### EXAMPLES

### Example I

### Preparation and Characterization of the Bioadsorbent

The aim of this study was to examine the capacity and efficacy of the receptor-based bioadsorbent. Relevancy was tested by treating plasma samples from diabetic and normal patients with the bioadsorbent and incubating the monocyte-derived (THP-1) cells with treated and non-treated samples. The effects of the samples on the regulation of pro-inflammatory and chemokine productions of THP-1 cells were then compared.

Preparation of Age-Modified Proteins. Age-Modified Bovine Serum Albumin (Age-BSA) was prepared by incubating BSA (˜50 mg/ml final; A-6003; Sigma-Aldrich, St. Louis, Mo.) in 1.67 M glucose dissolved in phosphate buffered saline pH 7.4 (0.01M PO₄, 0.138 M NaCl, 0.0027 M KCl), at 37° C. and 200 units of penicillin-streptomycin for two months under sterile conditions. Purified AGE-BSA (10 mg/ml) was also purchased from Research Diagnostic Inc (now RDI Division of Fitzgerald Industries Intl., Concord, Mass.) (AGE-BSA-RDI) and used to determine the equilibrium dissociation constant.

Protein expression and purification. The RAGE-His construct was a generous gift from Dr. Rosemarie Wilton at Argonne National Laboratory, Naperville, Ill. The RAGE-His construct was synthesized as disclosed in Wilton R., et al. (Wilton R., et al. (2006) Protein Expression Purification 47: 25-35; herein incorporated by reference in its entirety).

Briefly, a polynucleotide encoding the extracellular portion of RAGE was cloned into the E. coli expression vector pASK40 having polylinkers as modified by Yuri Londer (Argonne National Laboratory, Argonne, Ill.). The I.M.A.G.E. cDNA clone (clone ID 4718076) containing the complete coding sequence for human RAGE (SEQ ID NO: 7 encoding SEQ ID NO: 8) was obtained from the American Type Culture Collection (ATCC, Manassas, Va.). The polynucleotide encoding the extracellular region of RAGE from amino acid residues 23 through 340 (SEQ ID NO: 9) was amplified using the polymerase chain reaction (PCR) using the following oligonucleotides (MWG Biotech, High Point, S.C.): 5′-CTGACCTATGCGGCCGCTGCTCAAAACATCACAGC-3′ (SEQ ID NO: 10) and 5′GACTGAATTCATCAGTGATGATGGTGATGGTGAGTTCCCAGCCCTGATCC-3′ (SEQ ID NO: 11). The resulting polynucleotide therefore incorporated a NotI restriction site (single underline in SEQ ID NO: 10) in the region equivalent to the N-terminal portion of the encoded polypeptide sequence and a six-histidine tag followed by two stop codons and an EcoRI restriction site (double underline in SEQ ID NO: 11) in the region equivalent to the C-terminal portion of the encoded polypeptide. PCR was performed using Pfu DNA polymerase (Stratagene, La Jolla, Calif.) following the manufacturer's protocol. The resulting 1004 bp fragment was digested with NotI and EcoRI (Promega, Madison, Wis.). The fragment was ligated in frame with the OrnpA signal sequence of pASK40 containing the modified polylinker; T4 ligase was obtained from GibcoBRL/Invitrogen (Carlsbad, Calif.). The recombinant clones were sequenced (performed by MWG Biotech, High Point, S.C.) to confirm identity of the RAGE extracellular domain insert. Plasmids were transformed into E. coli strain JM83 for expression; bacterial stocks were maintained at −80° C. in LB medium containing 100 μg/ml carbenicillin and 15% glycerol.

Cells were streaked from a frozen glycerol stock of the RAGE-His construct onto LB/carbenicillin agar plates and grown overnight at 37° C. The next morning, colonies were recovered from the plate by washing with 2-3 ml of 2×TY media using a sterile cell scraper to loosen the colonies. The suspension was transferred to 1000 ml of 2×TY media containing 100 μg of carbenicillin. The culture was grown at 30° C., 250 rpm in an orbital shaker until the OD₆₀₀ was between 0.8-1.0. The culture was induced with 1 mM IPTG (isopropyl-beta-D-thiogalactopyranoside) and grown overnight (approximately 16-18 hours). The cells were harvested by centrifugation (8,000 rpm for 20 minutes) and stored at −80° C. Immediately, prior to purification, the cells were resuspended in 50 ml of ice cold TES (0.2 M Tris-HCl, 0.5 mM EDTA, 0.5 M Sucrose, pH 8.0) per liter of culture and incubated on ice for 20 minutes with gentle shaking and centrifuged for 20 minutes at 15,000 rpm. The supernatant was carefully removed to avoid any carryover of EDTA to the His affinity column during the purification step. The cells were resuspended in 40 ml of periplasmic buffer (5 mM MgSO₄, 20 mM Tris, pH 8.0), to which 300 μl of a protease inhibitor cocktail (Sigma P8849) and 300 μl of lysozyme buffer (20 mg/ml in TES, prepared fresh) were also added. The suspension was incubated on ice for 60 minutes with gentle shaking and centrifuged 20 minutes at 15,000 rpm to collect the periplasmic fraction. The periplasmic fraction was purified using a 5 ml bed volume HISTRAP HP affinity column, (Amersham Biosciences). The column was equilibrated with 10 volumes of Buffer A (20 mM TrisCl, 300 mM NaCl, 10 mM Imidazole). The periplasmic fraction was loaded onto the resin at a flow rate of 2.0 ml/min and run at a gradient of 0-60% Buffer B (20 mM TrisCl, 300 mM NaCl, 500 mM Imidazole). The peak fractions were identified on SDS-PAGE gel. The identified RAGE fractions were pooled; filter sterilized and dialyzed into 2,000 ml of HEPES buffer (10 mM HEPES, 150 mM NaCl, 3 mM EDTA, 0.005% Tween 20) overnight at 4° C. The dialysis buffer was changed the following day and the peak fractions (RAGE) were dialyzed again overnight at 4° C. in 2,000 ml of HEPES buffer.

Protein immobilization. RAGE was immobilized following a technique that was previously reported (Daniels, C. M., et al., (2005) Blood Purif., 23(4): 287-297, incorporated herein by reference in its entirety) except that a HEPES (10 mM HEPES, 150 mM NaCl, 3 mM EDTA, 0.005% Tween 20) buffer was used instead of PBS.

Horseradish peroxidase conjugation (HRP) of AGE-BSA. An HRP conjugation kit for labeling antigens and antibodies was purchased from Alpha Diagnostic International (San Antonio, Tex.) and used to label AGE-BSA (AGE-BSA-HRP) after the manufacturers' protocol.

Anti-AGE mouse monoclonal antibody. An anti-AGE mouse monoclonal antibody was purchased from Research Diagnostics Incorporation (Flanders, N.J.) and diluted to a final concentration of 0.25 μg/ml following the manufacturers' protocol.

Characterization of AGE-BSA. A competitive ELISA was developed and used to determine the AGE-BSA-adsorption site density or binding capacity of the agarose-immobilized RAGE. Four 1.5 ml centrifuge tubes were each loaded with 1 ml of PBS spiked with 30 μg/ml AGE-BSA and 25 μL of agarose-immobilized RAGE. The tubes were equilibrated at 37° C. for 1 hour in a Thermomixer R incubator shaker (Eppendorf/Brinkmann Instruments, Westbury, N.Y.) and then centrifuged. A calibration curve was generated and used to determine the amount of desorbed AGE-BSA. Antigen binding was determined using the following equation: ρₛ=Vₜₒₜₐₗ/V_{gel} (C₀−C_{eq}), where ρₛ is the adsorption site density (mg of AGE-BSA per ml of settled gel), Vₜₒₜₐₗ is the total volume (liquid+gel) of each tube (ml), V_{gel} is the settled-volume of the gel (ml), C₀ is the initial AGE-BSA concentration (mg/l), and C_{eq} is the concentration of desorbed AGE-BSA at equilibrium (mg/l).

Thermal stability of the bioadsorbent. Thermal stability was assessed by determining the binding capacity of AGE-BSA before and after incubating the bioadsorbent for four hours at 37° C. following a previously reported technique (Daniels (2005) supra).

Regeneration capacity of the bioadsorbent. The regeneration capacity was assessed by measuring the binding capacities before and after stripping the agarose-immobilized RAGE with 0.3 M Glycine buffer, pH 2.8 following a previously reported protocol (Daniels (2005) supra).

Storage in 20% ethanol. The bioadsorbent was stored in 20% ethanol for up to 34 days. Prior to assessing the binding capacity, the immobilized RAGE beads were centrifuged and the supernatant ethanol solution removed with a pipette. The beads were subsequently rinsed with PBS. Samples were assayed for AGE-BSA binding capacity at days 1, 3, 22, 31, and 34 (N=4 for each time point).

Combined effects of regeneration, ethanol storage, and temperature on the bioadsorbent. To mimic the conditions the immobilized RAGE would encounter during dialysis, the bioadsorbent was exposed to 6 cycles of clinically relevant operating conditions. Cycle 1 is the initial AGE-BSA binding capacity after storage in PBS at 4° C. Cycles 2-6 included the following: 1) regeneration and storage in 20% ethanol for 24 to 48 hours (RE). The bioadsorbent was then rinsed with PBS immediately prior to the determination of binding capacity (incubation for 1 hour at 37° C. in the presence of 30 μg/ml of AGE-BSA), and 2) regeneration, storage in 20% ethanol for 24 to 48 hours, and incubation for 3 hours at 37° C. followed by an additional 1 hour incubation in the presence of 30 μg/ml of AGE-BSA (RET). Prior to the next cycle, the bioadsorbent was regenerated, stored in 20% ethanol at 4° C., and rinsed with PBS. This procedure was repeated three times a week for two consecutive weeks. After each cycle, the AGE-BSA binding capacity was determined and compared to the initial value.

Lyophilization of the bioadsorbent. The bioadsorbent was frozen by placing in a −80° C. freezer overnight. The following day, the immobilized RAGE was lyophilized using a LABCONCO freeze drier system (LABCONCO Corporation, Kansas City, Mo.). The freeze-dried sample was reconstituted after storing for one week at room temperature with UV/UF water. To confirm that the integrity of the beads had not been compromised with the freeze drying process, photographs of the reconstituted and native (non-freeze-dried) bioadsorbent were taken using a Nikon Inverted Microscope, Eclipse TE2000-U (Nikon Instruments Inc., Melville, N.Y., USA) and the morphology of the beads were visually compared. Adsorption isotherm.

Equilibrium dissociation constant (K_{D}).The antigen-antibody equilibrium dissociation constant K_{D} was determined as previously described by Grovender et al. 2002 (Grovender, E. A., et al., (2002) supra). Briefly, the AGE-BSA binding capacity of the bioadsorbent was determined by equilibrating samples with different concentrations of AGE-BSA starting with subsaturating quantities (2.5, 5, 7.5, 10, 12.5, 15, 20, 25 30 μg/ml). Each reaction was incubated in PBS for 1 hour at 37° C. with the bioadsorbent. The supernatant was removed and the amount of protein in the samples was analyzed using the Bradford Assay and antigen binding was characterized. The value of the K_{D} was regressed by minimizing the sum-squared error (SSE).

Human plasma samples. Human plasma (HP) samples were obtained from twelve diabetic patients that were on dialysis and twelve healthy patients. Informed consent was obtained from all study participants. Samples were stored at −20° C. prior to analysis. The samples from the diabetic and healthy patients were diluted 1:4 (HP:PBS). Four tubes, each containing 25 μl of the bioadsorbent were incubated for 1 hour at 37° C. with 1 ml of the 1:4 dilution in a Thermomixer R incubator shaker. The results of the treated plasma from diabetic patients were compared to the original concentrations of AGEs in the non-treated samples and to the concentrations of AGEs in the plasma from normal patients.

Characterization of AGE removal in human samples. A competitive ELISA was used to determine the AGE removal potential of the bioadsorbent as described above.

Characterizing the removal of a range of pathological concentrations of AGE-BSA. Increasing concentrations of AGE-BSA (0.234, 0.938, 1.88, 3.75, 15, and 30 μg/ml) were incubated with PBS and incubated at 37° C. for one hour in a Thermomixer R incubator shaker and AGE removal was compared to non-treated samples using a competitive ELISA as described above.

THP-1 cell culture. Human monocytic THP-1 cells were purchased from ATCC (TIB 202, Maryland, USA), and cultured in RPMI 1640 containing 10% (v/v) fetal calf serum, 100 U/ml penicillin/streptomycin (Gibco BRL, Gaithersberg, Md., USA) and 50 μM mercaptoethanol at 37° C. and 5% CO₂. Approximately 1,000,000 THP-1 cells were incubated for 18 hours with 30 μg/ml of treated (T-ABSA) and non-treated AGE-BSA (NT-ABSA) and 200 μl of treated (T-Diabetic) and non-treated (NT-Diabetic) plasma from diabetic patients and 200 μl of normal human plasma (NHP). LPS (10 μg/ml) was used as a positive control stimulus. THP-1 cells incubated without any stimulus was used as a negative control (NC). After 18 hours at 37° C. and 5% CO₂, THP-1 cells were sedimented by centrifugation and the supernatant was removed and stored at −20° C. for subsequent analysis for cytokine release.

Cytokine release by THP-1 cells. Human IL-1β, Human IL-6, Human TNF-α/TNFSF1A, and IL-8 QUANTIKINE ELISA kits were purchased from R&D Systems (Minneapolis, Minn., USA). All experiments were carried out according to the manufacturer's protocol. The plates were read by a Tecan SAFIRE multifunctional microplate reader (Tecan Austria G.M.B.H) using an excitation and emission wavelength of 450 and 540 nm respectively.

Statistical analysis. Statistical analyses were performed using GRAPHPAD PRISM software package (GrapPad Software, San Diego Calif.). All experiments were performed in triplicates. Statistical significance was determined via one-way ANOVA test was used and p<0.05 was considered as statistically significant.

### Results

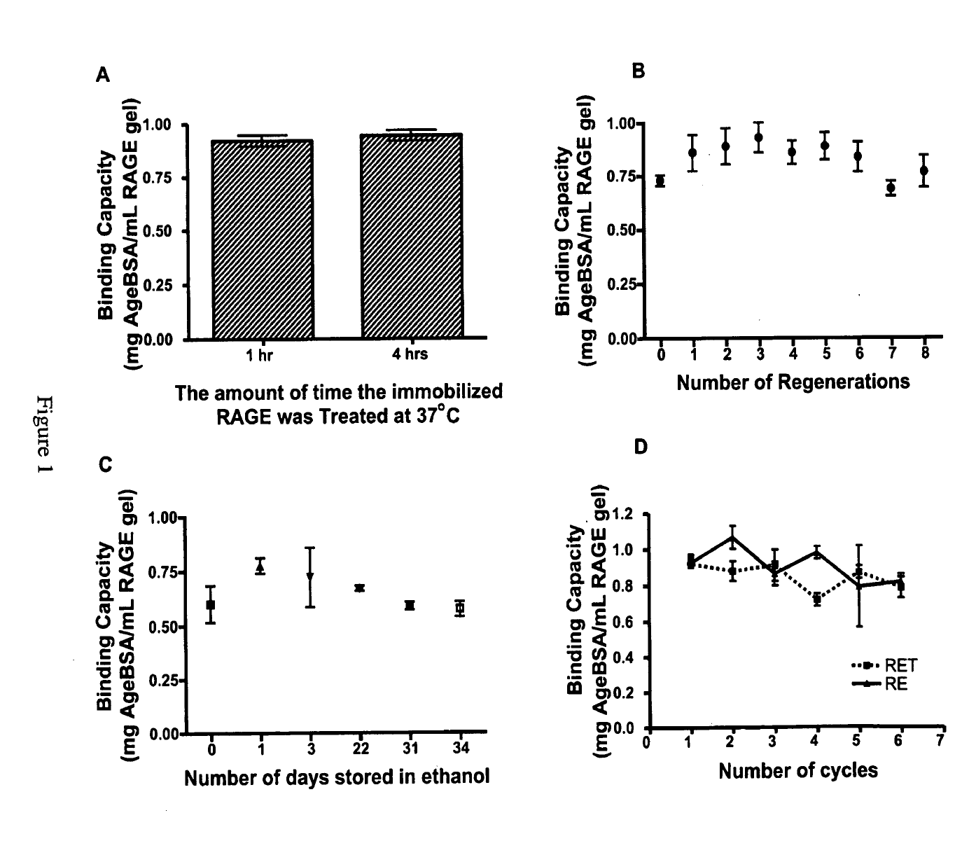

FIG. 1. (A) Incubating the bioadsorbent with saturating amounts of AGE-BSA for 1 and 4 hours at 37° C. tested thermal stability. The initial (0.92±0.08 mg AGE-BSA/ml RAGE gel) and final binding capacities remained constant (0.94±0.09 mg AGE-BSA/ml RAGE gel) throughout the treatment. (B) The regeneration capacity of the bioadsorbent was measured by determining the binding capacities before and after stripping the bioadsorbent with glycine buffer. After eight regenerations, the binding capacity of AGE-BSA remained relatively unchanged (p>0.05). (C) Storage of the bioadsorbent for 34 days in 20% ethanol at 4° C. did not have an effect on the AGE-BSA binding capacity, as the final binding capacity (0.57±0.06 mg AGE-BSA/ml RAGE gel) is not statistically different (p>0.05) from the initial binding capacity (0.6±0.17 mg AGE-BSA/ml RAGE gel). (D) The results from mimicking the conditions the bioadsorbent would encounter under clinical conditions. Although the final binding capacity (0.79±0.1 mg AGE-BSA/ml RAGE gel) decreased approximately 16% compared to the initial binding capacity (0.94±0.09 mg AGE-BSA/ml RAGE gel), exposure to regeneration, storage in 20% ethanol and 37° C. for 4 hrs (RET) did not significantly affect the AGE-BSA binding capacity of the bioadsorbent after 6 processing cycles (p>0.05). Similarly, subjecting a batch of the bioadsorbent to regeneration and storage in ethanol (RE) prior to incubation at 37° C. for 1 hour did not have a negative impact (p>0.05) on the AGE-BSA binding capacity as the final binding capacity (0.82±0.06 mg AGE-BSA/ml RAGE gel) decreased approximately 10% from the initial value (0.92±0.08 mg AGE-BSA/ml RAGE gel). The equilibrium adsorption behavior of AGE-BSA was determined using the Langmuir isotherm (Grovender, E. A., et al. (2002) supra). The dissociation constant was measured as being 46 nM. These results were consistent with the values obtained by others Neeper, M., et al., (1992) supra; Esposito, C., et al., (1989) J. Exp. Med., 170(4): 1387-1407; Schmidt, A. M., et al., (1992) J. Biol. Chem., 267(21): 14987-14997; Yang, Z., et al., (1991) J. Exp. Med., 174(3): 515-524; Skolnik, E. Y., et al., (1991) J. Exp. Med., 174(4): 931-939). All experiments were performed in quadruplicates (N=4) except for the experiments used to determine thermal stability (N=12) and the results are reported as the mean±SD.

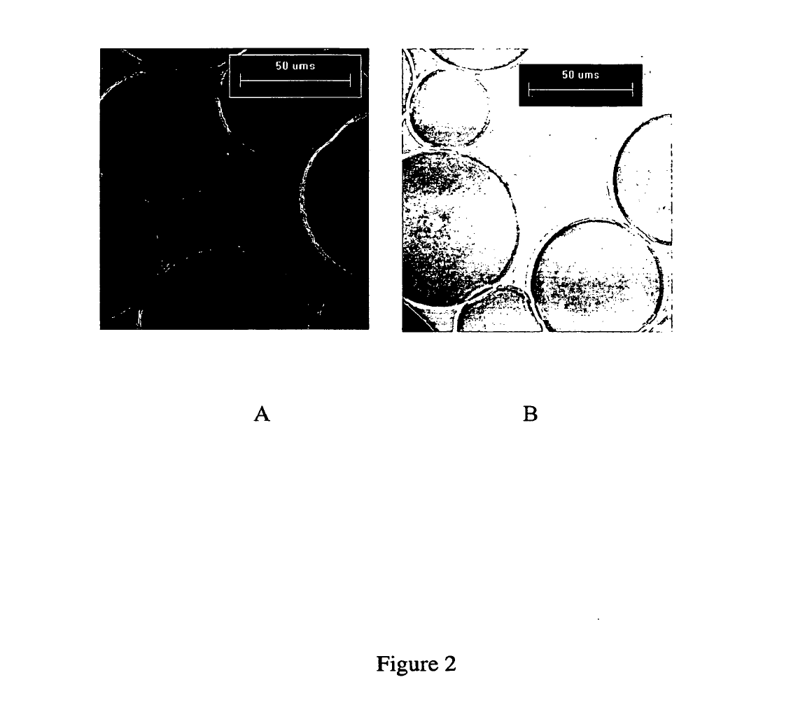

FIG. 2. To determine if the bioadsorbent can be stored long-term as a lyophilized product, the morphology of the reconstituted product was analyzed using light microscopy. (A) The reconstituted lyophilized beads maintained their spherical morphology compared to the (B) native beads suggesting the integrity of the beads remained intact after freeze-drying and lyophilization is adequate for long-term storage. Binding capacity after freeze-drying was also not affected by the process.

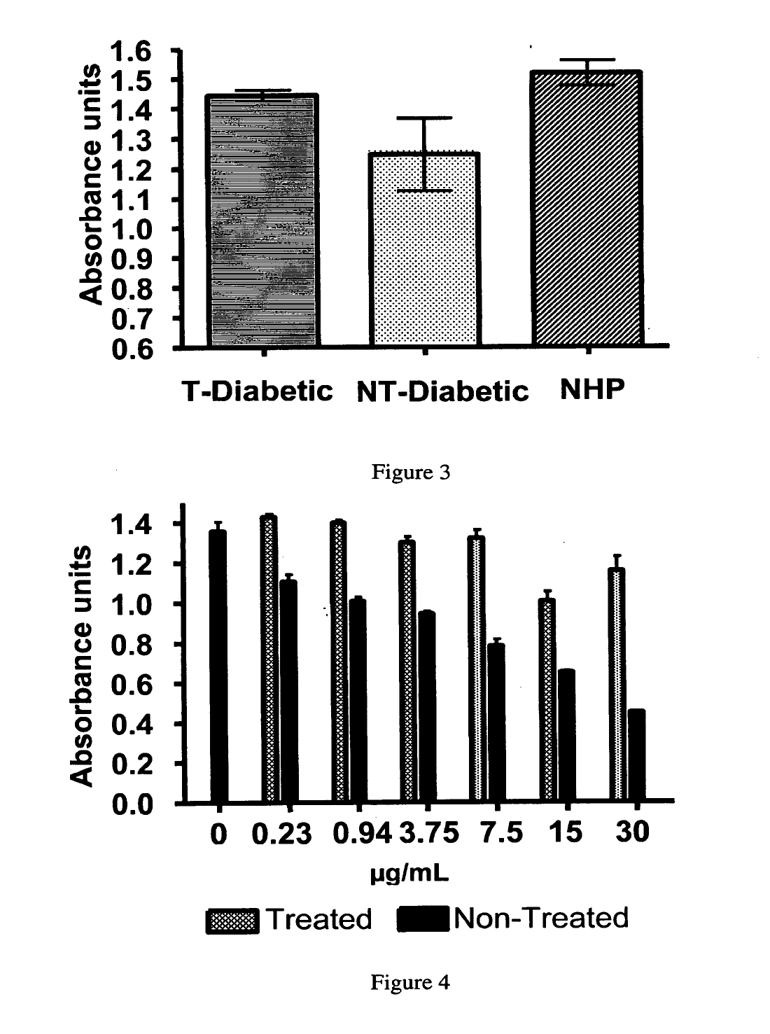

FIG. 3. The bioadsorbent was incubated in plasma separated from whole blood to mimic in vivo conditions. Plasma from diabetic and normal patients was diluted (1:4 Plasma/PBS) and incubated with the bioadsorbent and characterized using a competitive ELISA. The amount of AGEs left in the diabetic sample based upon the higher absorbance unit (1.45±0.02 AU) after treatment at 37° C. for one hour with the bioadsorbent was significantly lower that the amount of AGEs left in the non-treated sample (1.25±0.27 AU) (p<0.05). Moreover, the amounts of AGEs left in the samples from diabetic patients that were treated with the bioadsorbent were comparable to the amounts measured in the plasma of normal patients (1.52±0.1 AU, p>0.05). Mean±SD (N=3).

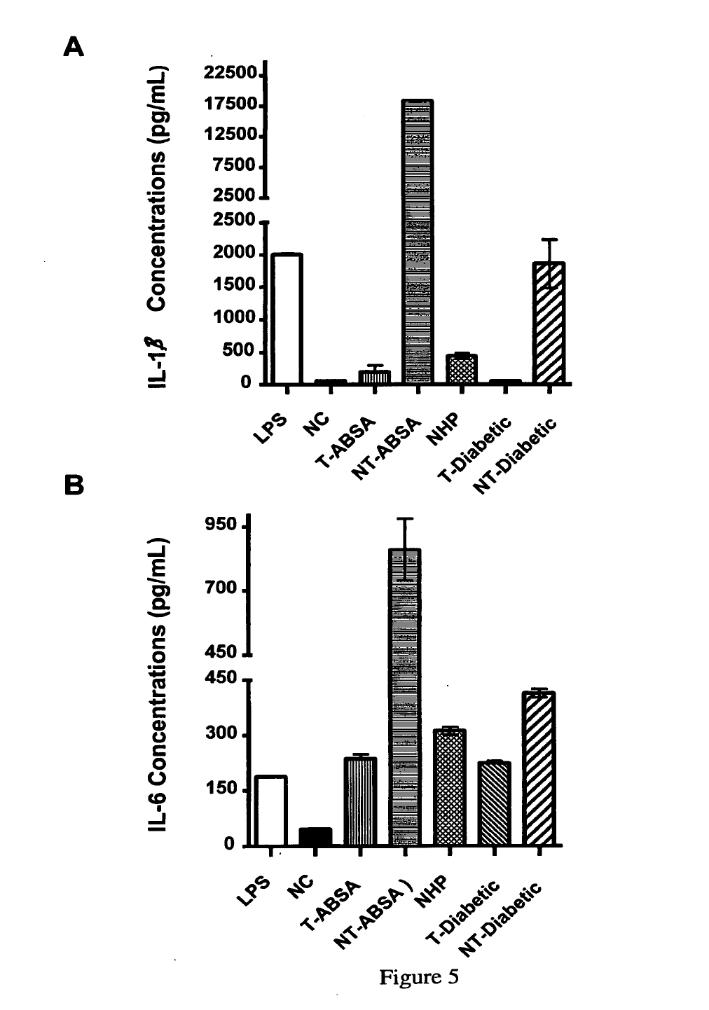

FIG. 4. The bioadsorbent was incubated with increasing amount of AGE-BSA to test a range of pathological concentrations of AGEs. The amount of AGEs in each sample was measured using a competitive ELISA and compared to the levels in the non-treated samples. As the amount of AGE-BSA increased in the spiked samples, a corresponding decrease was observed in the absorbance. When the samples were subsequently treated with the bioadsorbent, the levels of AGEs in each sample returned to normal levels. Mean±SD (N=3).

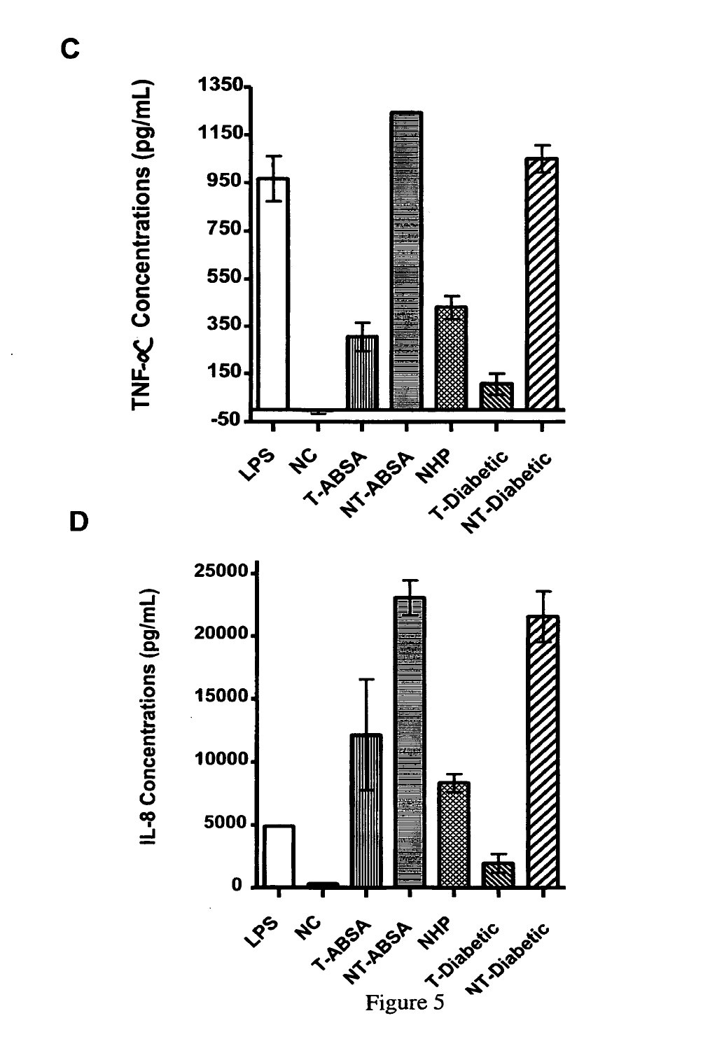

FIG. 5. Since others have shown through direct stimulation of a human monocytic cell line (THP-1 cells) with AGEs that cytokine production is upregulated (Yates, S. L., et al., (2000) J. Neurochem., 74(3): 1017-10125; Cheneval, D., et al., (1998) J. Biol. Chem., 273(28): 17846-17851), we investigated whether AGE-BSA and diabetic human samples treated with the bioadsorbent would downregulate the secretion of the pro-inflammatory cytokines such as IL-1β, IL-6, TNF-α, and the chemokine IL-8.

Cytokine and chemokine release are downregulated in THP-1 cells incubated with all glycated samples treated with the bioadsorbent. (A) IL-1β release by THP-1 cells incubated with T-ABSA and T-Diabetic elicited a significantly lower inflammatory response compared to non-treated samples (NT-ABSA and NT-Diabetic, p<0.001). Moreover, the treated samples were able to downregulate cytokine production in a manner comparable to the response observed from the stimulation by NHP (p>0.05). (B) IL-6 release by THP-1 cells stimulated with T-ABSA is significantly lower than NT-ABSA (p<0.001) and the decrease in stimulation is comparable to the stimulation by NHP (p>0.05). Although a decrease in IL-6 stimulation was observed between T-Diabetic compared to NT-Diabetic, and between T-Diabetic compared to NHP, these differences are not significant (p>0.05). (C) The release of TNF-α induced by all treated samples was less than that induced by non-treated samples (P<0.001). TNF-α stimulation by T-Diabetic was downregulated compared to NHP(P>0.01), and the decrease in TNF-α release by T-ABSA is comparable to what was observed by NHP (p>0.05). (D) IL-8 release by all the treated samples is lower than non-treated samples (p<0.001). The response elicited by T-Diabetic compared to NHP was significantly lower (p<0.05), whereas the level of stimulation by T-ABSA was comparable to the levels observed by NHP (p>0.05). All of the results are expressed as the means±SD (N=3).

### Example II

### Synthesis of Immobilized Receptor

The AGE-binding activity of a polypeptide was bound to a substrate following the method as disclosed by Daniels et al. (2005) (Daniels, C. M., et al. (2005) Blood Purif. 23: 287-297; herein incorporated by reference in its entirety).

Protein Expression and Purification. The polypeptide was expressed in a YVH10 yeast strain of Saccharomyces cerevisiae and purified via nickel affinity chromatography. The details of the protein expression and purification have been previously described herein.

Protein Immobilization. Increasing polypeptide densities were immobilized onto to a 1.5 ml settled volume of swollen SEPHAROSE CL4B (Amersham Biotech) using the cyanogen bromide chemistry (Pierce, Rockford, Ill., USA) for surface activation. The average diameter of the beads is 90 μm and the pore size of the beads allows a diffusion of molecules having a relative molecular mass of 6×10⁴ to 2×10⁷ into the inner pores of the beads. Immediately prior to the immobilization, the agarose support was washed extensively with deionized water and resuspended in an equal volume of deionized water. Two volumes (1.5 ml of agarose is equivalent to 1 volume) of a 2 M sodium carbonate buffer, pH 8 were added to 1 volume of the agarose support in PBS pH 7.4 (0.01 M PO4, 0.138 M NaCl, 0.0027 M KCl) and chilled on ice for 20 min. To increase the density of immobilized polypeptide on agarose beads, increasing concentrations of cyanogen bromide (0.3, 0.6, 0.9, 1.2, 1.5 and 1.8 g) dissolved in acetonitrile were added to the chilled agarose support and stirred vigorously for 5-7 minutes until the agarose was clear of any undissolved cyanogen bromide. The activated agarose was washed sequentially with the following ice cold buffers: 30 vol of 1 mM HCl, 30 vol of deionized water, 20 vol of NaHCO3 buffer (0.1 M NaHCO3, 0.5 M NaCl, pH 8.5) and 20 vol of PBS. Immediately thereafter, the polypeptide was added to the functionalized agarose gel and the immobilization reaction was carried out for 48 h on a rocker at 4° C. The unbound polypeptide was removed by centrifugation and the agarose-immobilized polypeptide was rinsed four times with 4 vol of PBS. The reaction was quenched with 4 vol of a lysine buffer (0.2 M lysine, 0.5 M NaCl, 0.1 M NaHCO3) and incubated overnight on a rocker at 4° C. The lysine buffer was removed and the agarose-immobilized polypeptide was rinsed four times with 4 vols of PBS. The lysine quenching buffer and the PBS rinses were assayed for protein leaching using the Bradford protein assay (BioRad, Hercules, Calif., USA) following the manufacturers' protocol.

Characterization. Four 1.5-ml centrifuge tubes were each loaded with 1 ml of fetal bovine serum solution FBS/PBS (75% FBS, 25% PBS) spiked with 30 μg AGE and 25 μA of agarose-immobilized polypeptide. AGE (AGE-BSA conjugate) was prepared as disclosed above. The tubes were equilibrated at 37° C. for 1 h in a THERMOMIXER R incubator shaker (Eppendorf/Brinkmann Instruments, Westbury, N.Y., USA) and then centrifuged. From each tube, 20 μl of a 6-fold dilution was loaded into individual wells of a 96-well plate, along with calibration standards. The plate was read by a Tecan SAFIRE multifunctional microplate reader (Tecan Austria G.M.B.H), using an excitation and emission wavelength of 450 and 620 nm, respectively. A calibration curve was generated and used to determine the amount of desorbed AGE. AGE binding was determined using the following equation: ρs=Vₜₒₜₐₗ/V_{gel} (C₀−C_{eq}), where ρs is the adsorption site density (mg of AGE per ml of settled gel), Vₜₒₜₐₗ is the total volume (liquid+gel) of each tube (ml), V_{gel} is the settled-volume of the gel (ml), C₀ is the initial AGE concentration (mg/l), and C_{eq} is the concentration of desorbed AGE at equilibrium (mg/l). The percentage of active polypeptide was calculated by dividing the actual adsorption site density by the theoretical adsorption site density if all of the immobilized polypeptide was available for binding AGE. The theoretical active polypeptide was calculated assuming a 1:1 RAGE polypeptide/AGE binding mole ratio.

Thermal Stability of the Immobilized Polypeptide. Thermal stability was assessed by determining the AGE binding capacity before and after incubating the immobilized polypeptide in FBS/PBS solution (75% FBS/25% PBS) for 5 h at 37° C. Four tubes, each containing 25 μl of immobilized polypeptide and 1 ml of FBS/PBS solution were incubated for 4 h at 37° C. After 4 h of incubation at 37° C., 30 μg of AGE was added to each tube and incubated for an additional hour after which the AGE binding capacity was assessed. The immobilized polypeptide was regenerated and stored in PBS at 4° C. until the following day. This procedure was repeated a total of three times.

Regeneration Capacity of the Immobilized Polypeptide. The regeneration capacity was assessed by measuring AGE binding capacity before and after stripping the agarose-immobilized polypeptide with 0.3 M glycine buffer, pH 2.8. Four tubes were assayed per regeneration. One volume of buffer is equivalent to 25 μl of immobilized polypeptide. Briefly, the immobilized polypeptide was regenerated by incubation with 40 volumes of stripping buffer for 1 min and rinsing four times with 40 volumes of PBS. This procedure was repeated until the immobilized polypeptide had been regenerated at least 20 times. The immunoadsorbent was stored in PBS at 4° C. during the time between regenerations.

Storage in Ethanol and Sterilization of the Immobilized Polypeptide. The immobilized polypeptide was stored in 20% ethanol for up to 39 days at 4° C. Prior to assessing the binding capacity, the immobilized polypeptide beads were centrifuged and the supernatant ethanol solution removed with a pipette. The beads were subsequently rinsed with PBS. Samples were assayed for AGE binding capacity at days 1, 4, 14, 25 and 39 (n=4 each time point). Also, the ability to regenerate ethanol-stored immobilized polypeptide was assessed by measuring the AGE binding capacity of immobilized polypeptide that had been stored in 20% ethanol and regenerated with glycine stripping buffer as described previously. After determination of the AGE binding capacity, the samples were regenerated, re-suspended in 20% ethanol, and stored in ethanol once again. This procedure was repeated with the same batch of immobilized polypeptide (n=4) at days 3, 9 and 44 of storage in ethanol at 4° C.

Sterility Test. Sterility was assessed using tryptic soy broth (TSB). TSB is a general purpose medium that is used for the cultivation of a variety of microorganisms and for sterility testing. After 1 day of storage in 20% ethanol at 4° C., the immobilized polypeptide was assessed by incubating 20 μl of supernatant from the immobilized polypeptide with 8 ml of TSB for 7 days at 35° C. The following controls were also incubated with TSB for 7 days at 35° C.: two negative controls (8 ml of TSB without sample and 8 ml of TSB incubated with 20 μl of 20% ethanol); three positive controls, 8 ml of TSB incubated with 20 μl of immobilized polypeptide stored in PBS at 4° C. with and without visible signs of contamination and 8 ml of TSB incubated with 20 μl of polypeptide secretion yeast.

Combined Effects of Temperature, Regeneration, and Ethanol Storage on the Immobilized Polypeptide. To mimic the conditions the immobilized polypeptide would encounter during dialysis, the immobilized polypeptide was exposed to 6 cycles of clinically relevant operating conditions, each cycle consisting of the following: (1) incubation in FBS/PBS solution for 4 h followed by a 5th hour in the presence of 30 μg of AGE; (2) regeneration of the AGE adsorption site density with glycine buffer, and (3) storage in 20% ethanol for 24-48 h at 4° C. Prior to the next cycle, the immobilized polypeptide was rinsed with PBS and the AGE binding capacity was determined. This procedure was repeated three times a week for two consecutive weeks. After each cycle, the AGE binding capacity was determined and compared to the initial value before treatment.

Lyophilization of the Immobilized Polypeptide. The immobilized polypeptide was frozen by placing in a −80° C. freezer overnight. The following day, the immobilized polypeptide was lyophilized using a LABCONCO freeze drier system (LABCONCO Corporation, Kansas City, Mo., USA). The freeze-dried sample was reconstituted after storing for one month at room temperature with UV/UF water. The reconstituted samples (25 μl) (n=4) were incubated with 1 ml of FBS/PBS spiked with 30 μg of AGE and equilibrated at 37° C. for 1 h in a Thermomixer R incubator shaker (1,400 rpm) as previously described. The supernatant was removed and AGE binding was determined. The reconstituted samples were tested for AGE and regenerated a total of five times. To confirm that the integrity of the beads had not been compromised with the freeze drying process, photographs of the reconstituted and native (non-freeze-dried) immobilized polypeptide were taken using a Nikon Inverted Microscope, Eclipse TE2000-U (Nikon Instruments, Melville, N.Y., USA) and the morphology of the beads were visually compared.

Statistical Significance. All experiments were performed in quadruplicates (n=4), except for the experiments used to determine the thermal stability of the immobilized polypeptide (n=12). The error estimates correspond to the accuracy of the gel volume measurement for the AGE binding capacity. Statistical significance between three or more means was determined via one-way analysis of variance (ANOVA), and the two sample t test was used to compare pairs of means. The criterion for statistical significance was a two-tailed p value of 0.05. The results were also compared to the currently accepted standards for the practice of reuse of dialyzers issued by the Association for the Advancement of Medical Instrumentation (AAMI). Their guidelines suggest that the total cell volume (TCV) should not fall below 80% of its original value. The TCV is equal to the volume of saline necessary to fill the blood compartment of the hemodializer. This value assures that the dialysis membrane would maintain a urea clearance of at least 90% of the original levels for small molecular weight molecules. Therefore a loss of AGE-binding capacity that is within 10% of the initial value before any regeneration is considered to be acceptable. Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1-18. (canceled)

19. A bioabsorbent comprising Receptor for advanced glycation endproducts (RAGE) immobilized onto CNBr-activated agarose, the immobilized RAGE having a binding capacity that remains relatively unchanged when incubated for up to 4 hours at 37° C.

20. The bioabsorbent according to claim 19, wherein the binding capacity of the bioadsorbent remains relatively unchanged after eight regeneration cycles, each regeneration cycle comprising regenerating the bioadsorbent by washing with a glycine buffer.

21. The bioabsorbent according to claim 19, wherein the binding capacity of the bioadsorbent remains relatively unchanged after storage for 34 days in 20% ethanol.

22. The bioabsorbent according to claim 19, wherein the binding capacity of the bioadsorbent remains relatively unchanged after eight regeneration cycles, each regeneration cycle comprising the steps of: (i) regenerating the bioadsorbent by washing the bioadsorbent with a glycine buffer; and (ii) storing the washed bioadsorbent in 20% ethanol for 24 to 48 hours.

23. The bioabsorbent according to claim 19, wherein the binding capacity of the bioadsorbent remains relatively unchanged after eight regeneration cycles, each regeneration cycle comprising the steps of: (i) regenerating the bioadsorbent by washing with a glycine buffer; (ii) storing the washed bioadsorbent in 20% ethanol for 24 to 48 hours; and (iii) incubating the washed bioadsorbent for 4 hours at 37° C.

24. The bioabsorbent according to claim 19, wherein the immobilized RAGE binds to advanced glycation endproducts (AGEs) reversibly.

25. The bioabsorbent according to claim 19, wherein the RAGE has an amino acid sequence selected from the group consisting of SEQ ID NO: 8 and SEQ ID NO: 9.

26. A system comprising the bioabsorbent of claim 19.

27. The system of claim 26, wherein the system is an extracorporeal system.

28. The system of claim 26, wherein the system is an in vivo implantable system.

29. A method for detoxifying blood from a subject in need of blood detoxification, the method comprising the steps of:
(i) providing a sample of fluid from a subject, the fluid comprising an AGE, the AGE having binding activity with the polypeptide;
(ii) incubating the sample with the system of claim 26 under appropriate binding conditions;
(iii) allowing the AGE to bind the immobilized RAGE and thereby deplete the sample of AGE; and
(iv) returning the AGE-depleted sample to the subject, thereby detoxifying the subject's blood.

30. The method of claim 29, further comprising the steps of:
(v) incubating the sample of fluid with isolated monocytes;
(vi) measuring the secretion of cytokine and chemokine from the monocytes; and
(vii) comparing the amount of measured cytokine and chemokine in the sample before depleting the AGE with the sample after depleting the AGE.

31. A method for detoxifying blood from a subject in need of blood detoxification, the method comprising the steps of:
(i) providing a sample of fluid from a subject, the fluid comprising an AGE, the AGE having binding activity with the polypeptide;
(ii) incubating the sample with a system comprising a polypeptide having receptor for advanced glycation endproducts (RAGE) activity and a substrate under appropriate binding conditions;
(iii) allowing the AGE to bind to the polypeptide and thereby deplete the sample of the AGE;
(iv) returning the AGE-depleted sample to the subject, thereby detoxifying the subject's blood;
(v) incubating the sample of fluid with isolated monocytes;
(vi) measuring the secretion of cytokine and chemokine from the monocytes; and
(vii) comparing the amount of measured cytokine and chemokine in the sample before depleting the AGE with the sample after depleting the AGE.

32. The method according to any one of claim 30 or 31, wherein the cytokine and chemokine are selected from the group consisting of interleukin 1 (IL-1), interleukin 2 (IL-2), interleukin 4 (IL-4), interleukin 5 (IL-5), interleukin 6 (IL-6), interleukin 7 (IL-7), interleukin 9 (IL-9), interleukin 10 (IL-10), interleukin 13 (IL-13), tumor necrosis factor α (TNF-α), interferon α (IFN-α), interferon α-II (IFN-α-II), interferon β (IFN-β), interferon γ (IFN-γ), interferon δ (IFN-δ), macrophage migration inhibitory factor (MIF), granulocyte-macrophage colony-stimulating factor (GM-CSF), granulocyte colony-stimulating factor (G-CSF), leukemia inhibitory factor (LIF), oncostatin (OSM), autocrine motility factor (AMF), lymphotoxin-α (LT-α), lymphotoxin-β (LT-β), T cell antigen gp39 (CD40L), CD27L, CD30L, FASL, 4-1BBL, OX40L, TNF-related apoptosis inducing ligand (TRAIL), platelet factor 4 (PF4), platelet basic protein (PBP), connective-tissue activating peptide III (CTAP III), (β-thromboglobulin, melanoma growth stimulatory activity protein (MGSA), macrophage inflammatory protein 2 (MIP-2), pre-B cell growth stimulating factor (PBSF), monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1 α (MIP-1-α), macrophage inflammatory protein 1 β (MIP-1-β), macrophage inflammatory protein 1 γ (MIP-1-γ), macrophage inflammatory protein 3 α (MIP-3-α), macrophage inflammatory protein 3 β (MIP-3-β), macrophage inflammatory protein 4 (MIP-4), macrophage inflammatory protein 5 (MIP-5), LD78 β, RANTES, SIS-epsilon (p500), thymus and activation-regulated chemokine (TARC), Eotaxin, I-309, human protein HCC-1/NCC-2, human protein HCC3, C-reactive protein (CRP), human PTX3, and chemotactic cytokine CP-10.

33. The method according to any one of claim 29 or 31, wherein the subject has a disease or disorder selected from the group consisting of diabetes, Alzheimer's disease, hemodialysis associated amyloidosis, and cardiovascular complications.

34. The method according to claim 31, wherein the substrate is selected from the group consisting of a micro-array, a particle, a porous particle, a membrane, a mesh, a dialysis membrane, a multi-well plate, a polymeric compound, wherein the polypeptide is chemically bound to the substrate.

35. The method according to claim 34, wherein the polymeric compound is agarose.

36. The method according to claim 31, wherein the polypeptide is selected from the group consisting of SEQ ID NO: 4, SEQ ID NO: 5, SEQ ID NO: 6, SEQ ID NO: 8, SEQ ID NO: 9, and a fragment thereof.
